(19) United States
(12) Patent Application Publication
(10) Pub. No.: US 2023/0301585 A1
(43) Pub. Date: Sep. 28, 2023

(54) Joint Evaluation

(71) Applicant: Howmedica Osteonics Corp., Mahwah, NJ (US)

(72) Inventor: Kevin Abbruzzese, Mahwah, NJ (US)

(21) Appl. No.: 18/121,704

(22) Filed: Mar. 15, 2023

Related U.S. Application Data
(60) Provisional application No. 63/323,195, filed on Mar. 24, 2022.

(51) Int. Cl. A61B 5/00; A61B 5/11; A61B 5/107

(52) U.S. Cl. CPC A61B 5/4585; A61B 5/1121; A61B 5/1071; A61B 2505/05

(57) ABSTRACT

Disclosed herein is a method for evaluation knee stiffness and knee damping. The method may include the steps of releasing a subject's leg from an extension position to allow the leg to oscillate under gravity, tracking a knee angle and at least one joint motion characteristic during the oscillation, determining a knee joint torque and a knee gravitational moment from the knee angle and the joint motion characteristic, and determining a knee stiffness and a knee damping from the joint torque and the knee gravitational moment.

### CROSS-REFERENCE TO RELATED APPLICATION

This application claims the benefit of the filing date of U.S. Provisional Patent Application No. 63/323,195 filed Mar. 24, 2022, the disclosure of which is hereby incorporated herein by reference.

### FIELD OF THE DISCLOSURE

The present disclosure relates generally to a method for joint evaluation, and in particular relates to a method for evaluating a knee joint.

### BACKGROUND OF THE DISCLOSURE

Joint movement evaluations for a total knee arthroplasty (“TKA”) are generally performed via subjective assessments utilizing imaging techniques, analog modalities, etc., to measure knee joint range of motion. These methods provide limited insight into dynamic biomechanical parameters on account of the subjective nature of the assessments. For example, knee joint movement evaluations such as the Lachman test, the anterior/posterior drawer test, the pivot shift test, the quadriceps active test, and the varus/valgus stress test, etc., generally fail to provide key information related to key knee joint characteristics such as stiffness, viscosity, damping, etc. Moreover, subjective evaluations depend on surgeon experience leading to inconsistent assessments.

Thus, an improved method for evaluating knee joints is desired.

### BRIEF SUMMARY OF THE DISCLOSURE

In certain embodiments, the present disclosure relates generally to a method for evaluating knee stiffness and knee damping.

In accordance with a first aspect of the present disclosure, a method to evaluate knee stiffness is provided. A method according to this aspect may include the steps of releasing a subject's leg from an extension position to allow the leg to oscillate under gravity, tracking a knee angle and at least one joint motion characteristic during the oscillation, determining a knee joint torque and a knee gravitational moment from the knee angle and the joint motion characteristic, and determining a knee stiffness from the joint torque and the knee gravitational moment.

Continuing in accordance with this aspect, the step of releasing a subject's leg from an extension position may include the step of allowing the leg to oscillate until the leg comes to rest in a flexion position. The step of tracking the joint angle and the joint motion characteristic may include the step of tracking the joint angle and the joint motion characteristic at multiple intervals or continuously throughout the oscillation. The step of releasing the subject's leg from an extension position may include the step of releasing the subject's leg from a joint angle of 0 degrees.

Continuing in accordance with this aspect, the joint angle may be tracked and recorded using sensors. The sensors may be any of a camera, a location sensor, an orientation sensor, a movement sensor, a proximity sensor, and a magnetic sensor. The at least one motion characteristic may be a joint angular velocity.

Continuing in accordance with this aspect, the knee gravitational moment may be determined from a mass of the leg and the joint angle. The method may be performed prep-operatively to determine a pre-operative knee stiffness. The method may be performed post-operatively to determine a post-operative knee stiffness. The method may include a step of comparing the pre-operative knee stiffness to the post-operative knee stiffness.

In accordance with a second aspect of the present disclosure, a method to evaluate knee stiffness is provided. A method according to this aspect may include the steps of releasing a subject's leg from an extension position to allow the leg to oscillate under gravity, tracking a knee angle and at least one joint motion characteristic during the oscillation, determining a knee joint torque and a knee gravitational moment from the knee angle and the joint motion characteristic, and determining a knee damping from the joint torque and the knee gravitational moment.

Continuing in accordance with this aspect, the step of releasing a subject's leg from an extension position may include the step of allowing the leg to oscillate until the leg comes to rest in a flexion position. The step of tracking the joint angle and the joint motion characteristic may include the step of tracking the joint angle and the joint motion characteristic continuously or at multiple intervals throughout the oscillation. The step of releasing the subject's leg from an extension position may include the step of releasing the subject's leg from a joint angle of 0 degrees.

Continuing in accordance with this aspect, the joint angle may be tracked using sensors. The sensors may be any of a camera, a location sensor, an orientation sensor, a movement sensor, a proximity sensor, and a magnetic sensor. The at least one motion characteristic may be a joint angular velocity.

Continuing in accordance with this aspect, the knee gravitational moment may be determined from a mass of the leg and the joint angle. The method may be performed prep-operatively to determine a pre-operative knee damping. The method may be performed post-operatively to determine a post-operative knee damping. The method may include a step of comparing the pre-operative knee damping to the post-operative knee damping.

### BRIEF DESCRIPTION OF THE DRAWINGS

A more complete appreciation of the subject matter of the present disclosure and the various advantages thereof may be realized by reference to the following detailed description, in which reference is made to the following accompanying drawings:

FIG. 1 is a flowchart showing a method to determine a knee stiffness according to an embodiment of the present disclosure;

FIG. 2 is a flowchart showing a method to determine a knee damping according to an embodiment of the present disclosure;

FIG. 3 is a graph showing a change in total knee moment, gravitational knee moment and net knee moment over time during a joint oscillation test;

FIG. 4 is a graph showing a net knee moment versus knee angle;

FIG. 5 is a graph showing a net knee moment versus knee angular velocity;

FIG. 6 is a graph showing a net knee moment versus knee angle for a graphical stiffness estimate, and

FIG. 7 is a graph showing a net knee moment versus knee angle for a graphical damping estimate.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described. As used herein, the terms “knee” and “joint” will be used interchangeably and as such, unless otherwise stated, the explicit use of either term is inclusive of the other term.

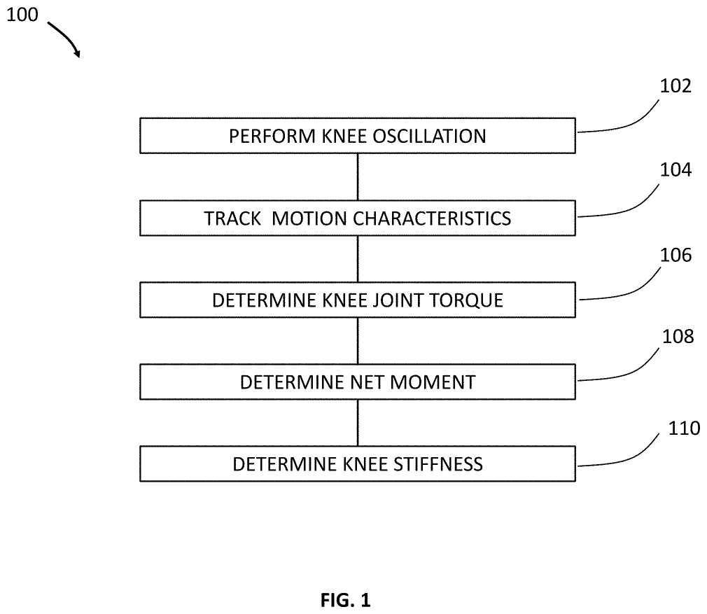

FIG. 1 is a flowchart 100 showing a method for determining knee stiffness according to an embodiment of the present disclosure. In a first step 102, an operator performs a knee oscillation test such as a pendulum knee drop test (“PKD”) on a patient's knee. The oscillation test is performed by positioning the patient's leg in near full extension, and releasing the leg. The leg is allowed to fall under gravity, and oscillate freely until the leg comes to rest in a flexion position. While an operator facilitates the performance of the oscillation test in this embodiment, the oscillation test can be performed by the patient with no assistance. Simultaneously with the oscillation test in step 102, a step 104 to measure various knee joint motion characteristics is performed. Various sensors such as tracking cameras, location sensors, orientation sensors, movement sensors, proximity sensors, load sensors, magnetic sensor, inertial measurement units (“IMUs”), etc. can be used to track and record the knee oscillation. The tracking and recording can be performed continuously or at multiple regular or irregular intervals. Sensors can be remote (such as cameras), internal (placed in implants, trials, or joint tensioners), or external (such as wearables) to collect joint motion characteristics such as knee angle, knee angular velocity, force between the tibia and femur, number of oscillations, oscillation time, etc.

The tracked joint motion characteristics in step 104 representing knee kinematics are determined by forces acting on the knee joint and represent the summation of knee joint torque (Iθ″) as shown in Equation 1 below.

Iθ″=K*θ+B*θ′+m*g*L_{com}*sin(θ)

Using the joint motion characteristics tracked in step 104 and Equation 1, knee joint torque can be estimated in a step 106. Here, “L_{com}” denotes a model of the knee acting with a mass (“m”), which is moved by a gravitational knee moment (g*sin θ). Knee stiffness (K) and knee damping (B) represent the intrinsic characteristics of the knee joint during the oscillation test. Knee damping (B) represents the knee joint dissipated energy or resistance offered by the knee joint to motion. Knee stiffness (K) represent stiffness due to intrinsic soft tissue or active musculature. “L_{com}” represents a proximal length of a patient's leg to a center of mass. L_{com} may be derived from anthropometric estimations, which may in turn be estimated from segment masses and known anthropometric constants. A total mass of the patient can be represented by “m.” A gravitational constant is represented by “g.” “θ” represents a flexion angle of the knee joint, “θ″” represents knee angular velocity, and “θ″” represents knee angular acceleration. Inertia “I” representing leg or foot segment mass can be estimated from known anthropometric estimations.

A knee net moment (Mₙₑₜ) of the knee representing a summation of the knee stiffness (K) and knee damping (B) components is computed in a step 108 using Equation 2 below.

Iθ″=Mₙₑₜ+m*g*L_{com}*sin(θ)

As shown in Equation 2, the total knee moment includes net knee moment (Mₙₑₜ) and gravitational knee moment. Iθ″ (sum of the moments) consists of the inertia of the knee and the angular acceleration. Both these properties can be computed from sensor information. For example, gravitational moment (m*g*L_{com}*sin(θ)) can be derived from measured knee angles (via a sensor) and constant parameters such as gravity, segment mass, and length of the leg. In a final step 110, knee stiffness (K) is computed from Equation 2 above.

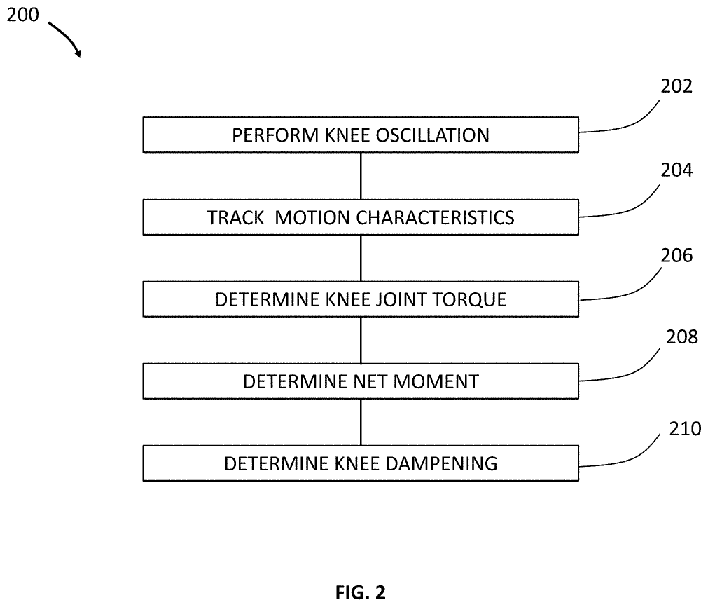

Referring now to FIG. 2, there is shown a flowchart 200 illustrating steps for determining a knee damping according to another embodiment of the present disclosure. Flowchart 200 is similar to flowchart 100, and therefore like method steps are referred to with similar numerals within the 200-series. For example, flowchart 200 includes a step 202 to perform a knee oscillation test, and a step 204 to track joint motion characteristics during the knee oscillation test. However, a knee damping value is computed in a final step 210 from Equation 2 in flowchart 200.

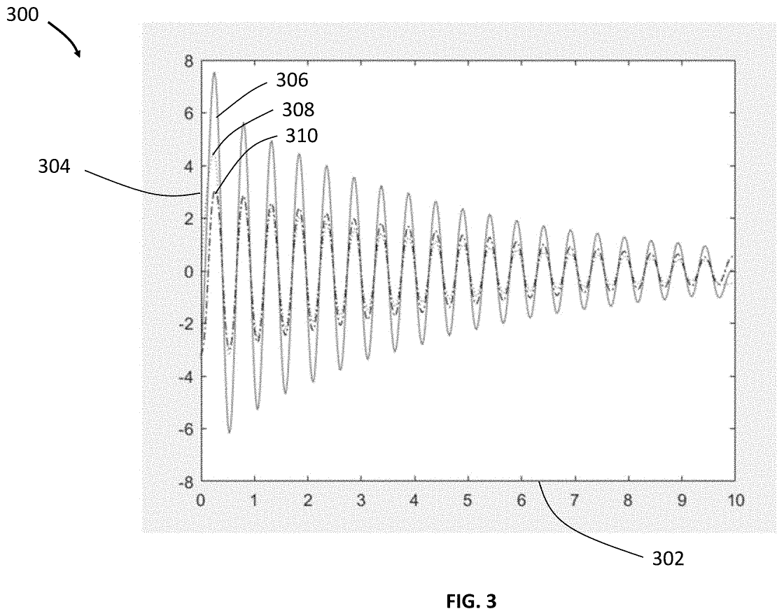

FIG. 3 is a graph 300 depicting time (s) 302 versus moment (N-m) 304 plotting a total knee moment 306, a net knee moment (Mₙₑₜ) 308, and a gravitational knee moment (g*sin θ) 310 captured by the one or more sensors during the knee oscillation test. Total knee moment 306 is the sum of net knee moment 308 and gravitational knee moment 310 as shown in FIG. 3. While net knee moment 308 is greater than gravitational knee moment 310 for the first few oscillations of the knee, eventually the gravitational knee moment exceeds the net knee moment before the knee comes to a full rest when the total knee moment 306 is 0 N-m at 10 s as shown in FIG. 3.

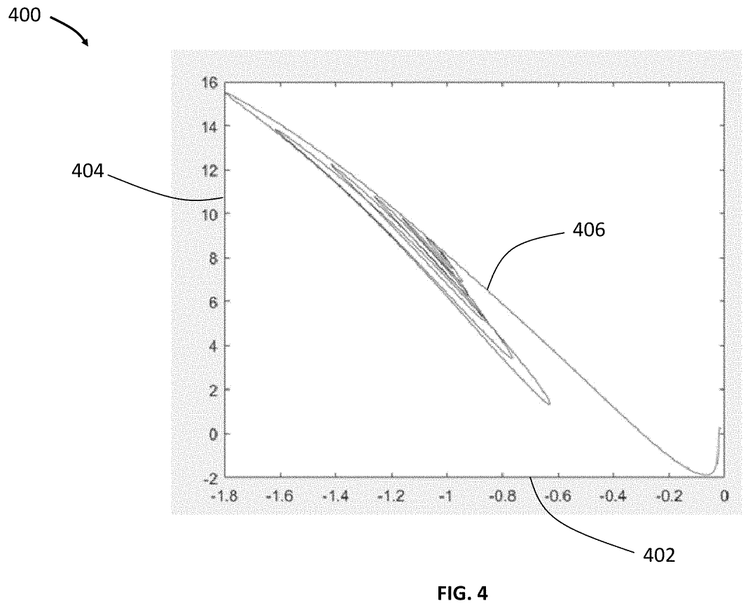

Referring now to FIG. 4, there is shown a graph 400 depicting knee angle (rad) 402 versus moment (N-m) 404 plotting a net knee moment (Mₙₑₜ) 406 measured by the one or more sensors during the knee oscillation test. The net knee moment 406 fluctuates between 16 N-m and −2 N-m as the knee angle (range of motion) varies from 0 radians to −1.8 radians, respectively, during the oscillation test. An instantaneous knee stiffness (K) value can be computed by calculating a slope of net knee moment 406 at any instant during the oscillation test.

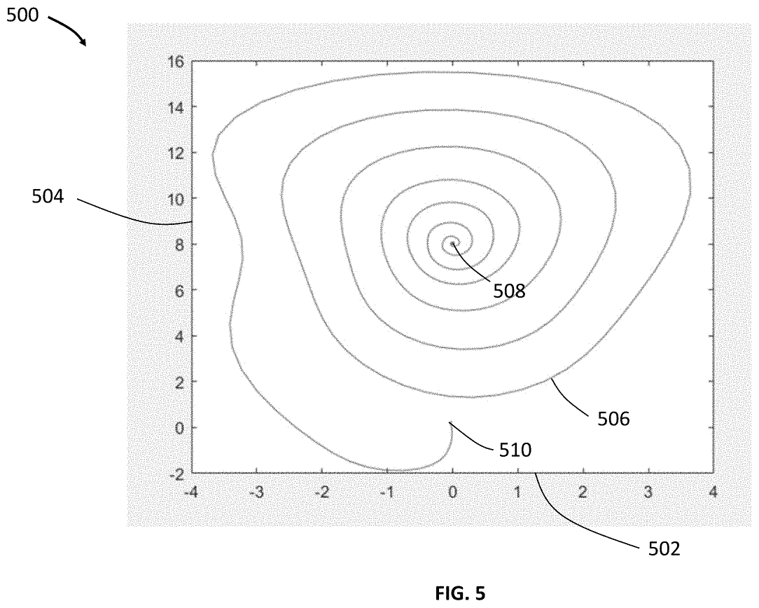

FIG. 5 is a graph 500 depicting knee angular velocity (rad/s) 502 versus moment (N-m) 504 plotting a net knee moment (Mₙₑₜ) 506 measured by the one or more sensors during the knee oscillation test. As shown in graph 500, the net knee moment 506 fluctuates between 16 N-m and −2 N-m as knee angular velocity fluctuates between 4 rad/s and −4 rad/s. At the start of the knee oscillation test, knee angular velocity is 0 as denoted by a marker 508. When the knee returns to a complete rest, knee angular velocity is once again 0 as shown by a marker 510. An instantaneous knee damping (B) value can be computed by calculating a slope of net knee moment 506.

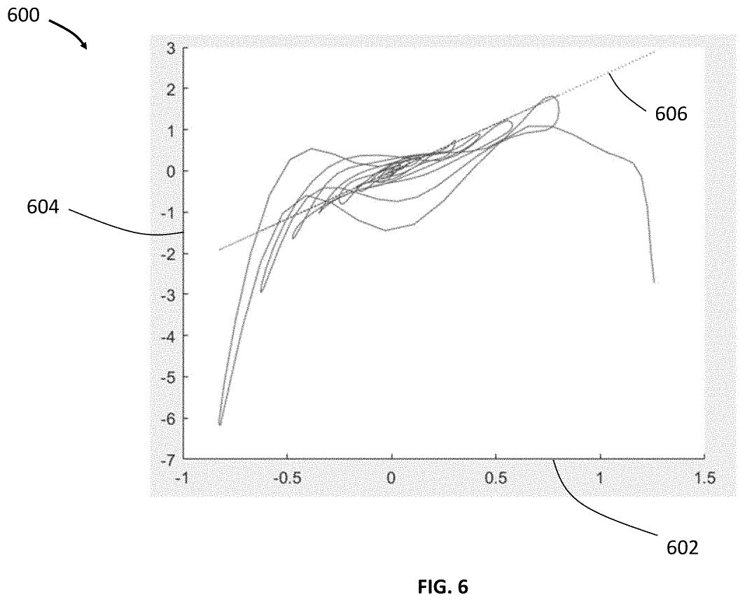

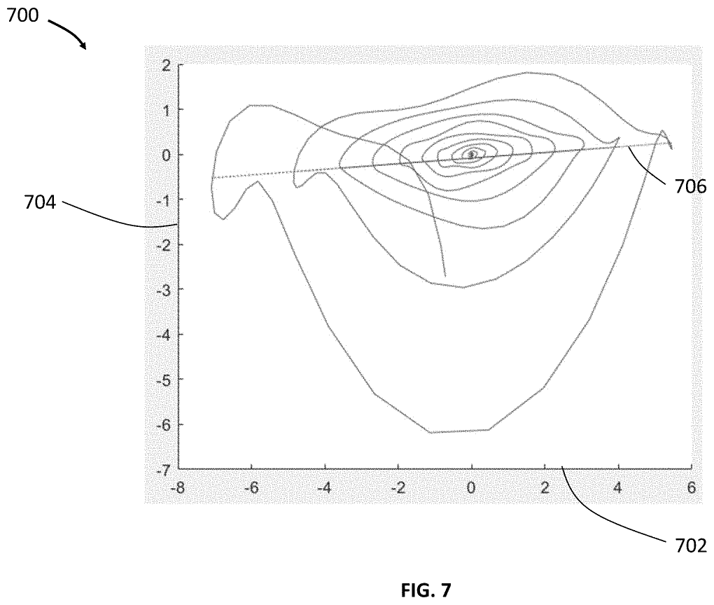

Rotational torque or moments of the knee joint can be influenced by stiffness (K) and damping factors (B). Moments as a result of stiffness are directly influenced by rotational angle and displacement. As rotational dampers are velocity-dependent parameters, stiffness (K) and damping (B) curves can be generated based on the summation of the moments. For example, FIG. 6 shows a graph 600 depicting net knee moment (Mₙₑₜ) 604 versus knee angle 602. A slope 606 derived from derived from a linear region of measured Mₙₑₜ and knee angle values represents a graphical estimate of stiffness coefficient (K) as shown in FIG. 6. FIG. 7 shows a graph 700 depicting net knee moment (Mₙₑₜ) 704 versus knee angle 702. A slope 706 derived from a central region of measured Mₙₑₜ and knee angle values represents a graphical estimate of damping coefficient (B) as shown in FIG. 7. Measurements or samples can be evaluated after 150 cycles to ensure that consistent oscillations with reduced amplitudes are achieved. Therefore, once Mₙₑₜ defined in Equation 2 is computed, Mₙₑₜ can then be used to provide a graphical estimation of the stiffness (K) and damping (B) coefficients by identifying the slope of the respective curves representing measured knee stiffness (K) and knee damping (B). In both cases, a best linear fit can be applied to estimate the slope of the curves to solve for these coefficients. Alternatively, an algorithm can be used to differentiate knee stiffness (K) and knee damping (B) values using a statistical model—for example, a goodness of fit model or the like, where the sum of squared error (“SSE”) is automatically generated to provide the smallest degree of error between the input curve and the model estimate.

In another embodiment, the Levenberg-Marquart algorithm (“LMA”) can be used to graphically estimate knee stiffness (K) and knee damping (B) can be graphically estimated. These estimates can serve as an input for a model, which can then be tuned and optimized to reduce the system error. A least squares nonlinear model utilizing graphical estimates can be used to compute differences between an input curve and the model curve until error values for stiffness (K) and damping (B) satisfy predetermined thresholds. Thus, knee stiffness (K) and knee damping (B) can be computationally estimated and optimized using these techniques.

The methods disclosed herein can be performed pre-operatively, intra-operatively and post-operatively. Knee stiffness and knee damping values can be obtained throughout a TKA process and stored for each patient to create a database. This information can complement robotically generated data or augment manual surgical procedures. Data collected at various stages of a TKA can be utilized for predictive analytics to potentially determine factors like implant selection, surgical technique for soft tissue, physical therapy prescriptions, correlations to patient reported outcome measures (PROMs), etc. Pre-operatively, an operator can utilize computed knee stiffness derived from the methods disclosed herein to aid in implant selection, screen patients for neurophysiological conditions, identity early intervention, etc.

Oscillation tests performed intra-operatively can be used to assist with soft tissue balance during TKA. An oscillation test performed with trial or implants placed intra-operatively can provide force loading on the medial and lateral condyles during knee oscillation. Specific loading conditions for medial and lateral compartments can be assessed throughout the intra-operative state in static and dynamic conditions using force measuring sensors located in tibial trials, joint tensioners, ligament balancers, etc. For static conditions, substitutions of tibial polyethylene trials of various fixed heights or joint tensioners can be used to displace the joint a fixed distance. Known displacements when combined with the force sensors can be used to create a force-displacement curve. The force-displacement curves can be generated for both the medial and lateral compartments on the knee joint to create independent force curves to estimate knee stiffness coefficients for the medial and lateral compartments. The slope or derivative of the force-displacement curves represents the overall knee stiffness which can be correlated to the either the medial or the lateral compartment. The stiffness values derived from the static assessment can be used to estimate overall laxity, with lax ligaments exhibiting a lower stiffness compared to less lax ligaments exhibiting higher stiffness. Alternatively, the overall work performed by each ligament can be calculated as the area under the force curve—i.e., the cumulative sum of the ligament force over the distance of displacement. The knee stiffness and work parameters can be estimated from force sensors at fixed displacements and compared to the global knee stiffness values obtained from the oscillation test as shown below in Equation 3.

Overall Knee Stiffness=Medial Knee Stiffness+Lateral Knee Stiffness+C

The medial and lateral stiffness represent the stiffness values obtained from the force sensors and fixed displacements. C represents a coefficient of a cumulative stiffness from characterized features like musculature, fluid, inflammation, ligaments, etc. Knee stiffness is the resistance to deformation or movement under a loading condition. As explained above, in either static or dynamic loading conditions, force data can be paired with displacement data or angular data to generate force-displacement curves that can be utilized to estimate cumulative or individual ligament laxities as knee stiffness values. The addition of the force sensors provides the ability to quantify individual compartments and collateral ligaments. Inclusion of force information allows for the estimation of work performed by each ligament in either a static or dynamic condition. The quantification of ligament laxity provides real-time feedback to an operator to perform additional laxity tuning through adjustment of either implant size, bony resection, or ligament resection. These changes can be quantified and offer the operator the ability to correct or adjust soft tissue tension targets to allow for improved outcomes for the patient. Patient specific data obtained from the force sensing device and motion capture platform can be used to tune ligament parameters that are ideal for the patient and maintain the appropriate degree of laxity. Optimized stiffness tuned in real-time can augment surgeon decision making and execution which may improve OR efficiencies and patient performance post-operatively.

Post-operative knee stiffness and damping can be used to evaluate patient recovery and identify inflammation, instability, faulty implant mechanics, etc. Knee stiffness and damping can be assessed repeatedly over time using the methods disclosed herein to track and document patient progress.

While a knee joint is described in the various methods disclosed herein, the present disclosure can be used for any other joint such as hip, shoulder, ankle, etc. Furthermore, although the embodiments disclosed herein has been described with reference to particular features, it is to be understood that these features are merely illustrative of the principles and applications of the present disclosure. It is therefore to be understood that numerous modifications, including changes in the sizes of the various features described herein, may be made to the illustrative embodiments and that other arrangements may be devised without departing from the spirit and scope of the present disclosure. In this regard, the present disclosure encompasses numerous additional features in addition to those specific features set forth in the paragraphs below. Moreover, the foregoing disclosure should be taken by way of illustration rather than by way of limitation as the present disclosure is defined in the examples of the numbered paragraphs, which describe features in accordance with various embodiments of the disclosure, set forth in the claims below.

1. A method to evaluate knee stiffness, the method comprising the steps of:
releasing a subject's leg from an extension position to allow the leg to oscillate under gravity;
tracking a knee angle and at least one joint motion characteristic during the oscillation;
determining a knee joint torque and a knee gravitational moment from the knee angle and the joint motion characteristic; and
determining a knee stiffness from the knee joint torque and the knee gravitational moment.

2. The method of claim 1, wherein the step of releasing a subject's leg from an extension position includes the step of allowing the leg to oscillate until the leg comes to rest in a flexion position.

3. The method of claim 2, wherein the step of tracking the knee angle and the joint motion characteristic includes the step of tracking the knee angle and the joint motion characteristic at multiple intervals throughout the oscillation.

4. The method of claim 3, wherein the step of releasing the subject's leg from an extension position includes the step of releasing the subject's leg from a joint angle of 0 degrees.

5. The method of claim 3, wherein the knee angle is tracked using sensors.

6. The method of claim 5, wherein the sensors are any of a camera, a location sensor, an orientation sensor, a movement sensor, a proximity sensor, and a magnetic sensor.

7. The method of claim 3, wherein the at least one joint motion characteristic is a joint angular velocity.

8. The method of claim 1, wherein the knee gravitational moment is determined from a mass of the leg and the knee angle.

9. The method of claim 1, wherein the method is performed prep-operatively to determine a pre-operative knee stiffness.

10. The method of claim 9, wherein the method is performed post-operatively to determine a post-operative knee stiffness.

11. The method of claim 10, further including a step of comparing the pre-operative knee stiffness to the post-operative knee stiffness.

12. A method to evaluate knee damping, the method comprising the steps of:
releasing a subject's leg from an extension position to allow the leg to oscillate under gravity;
tracking a knee angle and at least one joint motion characteristic during the oscillation;
determining a knee joint torque and a knee gravitational moment from the knee angle and the joint motion characteristic; and
determining a knee damping from the knee joint torque and the knee gravitational moment.

13. The method of claim 12, wherein the step of releasing a subject's leg from an extension position includes the step of allowing the leg to oscillate until the leg comes to rest in a flexion position.

14. The method of claim 13, wherein the step of tracking the knee angle and the joint motion characteristic includes the step of tracking the knee angle and the joint motion characteristic at multiple intervals throughout the oscillation.

15. The method of claim 14, wherein the step of releasing the subject's leg from an extension position includes the step of releasing the subject's leg from a joint angle of 0 degrees.

16. The method of claim 14, wherein the knee angle is tracked using sensors.

17. The method of claim 16, wherein the sensors are any of a camera, a location sensor, an orientation sensor, a movement sensor, a proximity sensor, and a magnetic sensor.

18. The method of claim 14, wherein the at least one motion characteristic is a joint angular velocity.

19. The method of claim 12, wherein the knee gravitational moment is determined from a mass of the leg and the knee angle.

20. The method of claim 12, wherein the method is performed prep-operatively to determine a pre-operative knee damping.
